(19) United States
(12) Patent Application Publication
(10) Pub. No.: US 2009/0099367 A1
(43) Pub. Date: Apr. 16, 2009

(54) Process for preparing a leukotriene antagonist

(75) Inventors: Jordi Bessa Bellmunt, Barcelona (ES); Llorenc Rafecas Jane, Llorenc del Penedes (Tarragona) (ES); Mireia Pasto Aguila, Barcelona (ES); Xavier Verdaguer Espaulella, Vic (Barcelona) (ES); Esther Gordo Campon, Barcelona (ES)

Correspondence Address: BERENBAUM, WEINSHIENK & EASON, P.C, 370 17TH STREET, SUITE 4800, DENVER, CO, 80202, US

(73) Assignee: FARMAPROJECTS, S. A., L'Hospitalet de Llobregat (Barcelona) (ES)

(21) Appl. No.: 12/281,947

(22) PCT Filed: Mar. 5, 2007
(86) PCT No.: PCT/EP07/52062

§ 371 Date: Sep. 5, 2008

(30) Foreign Application Priority Data
Mar. 6, 2006 (EP) 06110729.8

(51) Int. Cl. C07D 215/18; C07C 321/20

(52) U.S. Cl. 546/174; 562/426

(57) ABSTRACT

Process for preparing montelukast or a pharmaceutically acceptable salt thereof, especially its sodium salt, that comprises the condensation of an aldehyde and 7-chloro-2-methylquinoline. Moreover, novel intermediates useful for the synthesis of montelukast are described as well as their preparation.

### FIELD OF THE INVENTION

The present invention relates to a process for preparing a leukotriene antagonist, in particular montelukast and salts thereof. It also relates to new intermediates useful in such process.

### BACKGROUND OF THE INVENTION

Montelukast sodium is a leukotriene antagonist of formula:

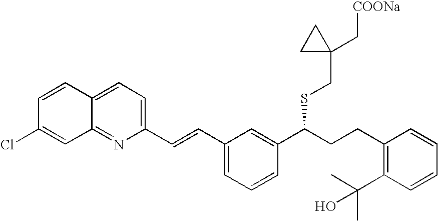

Montelukast sodium is also known as sodium R-(E)-1-[[[1-[3-[2-[7-chloro-2-quinolinyl]ethenyl]phenyl]-3-[2-(1-hydroxy-1-methylethyl)phenyl]propyl]thio]-methyl]cyclopropaneacetate. This compound is useful in the treatment of asthma, inflammation, allergies, angina, cerebral spasm, glomerular nephritis, hepatitis, endotoxemia, uveitis and allograft rejection.

Montelukast and montelukast sodium salt were first disclosed in EP480717-A1. In this document several synthetic routes were described for the final steps of the synthesis of montelukast, salts thereof, structurally related compounds and intermediates. The synthesis of montelukast sodium that was described in Example 161 could be included in the following general strategy (A).

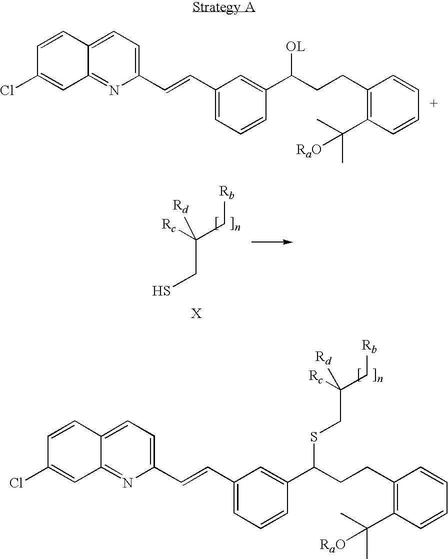

wherein:
L is an alcohol activating group,

Ra is hydrogen or an alcohol protecting group,

Rb is carboxylic acid, its salts or an intermediate or protected form, such as ester, amide, cyano, etc.
Rc, Rd are hydrogen or alkyl or Rc and Rd may form a cycloalkane, e.g. cyclopropane, and
n is 0 or 1.

The thiol intermediate X can also be in the form of an alkaline thiolate salt.

In Example 161 of EP480717-A1, L is methanesulfonyl; Rₐ is tetrahydropyranyl (THP); R_{b} is COOMe and R_{c}-R_{d} together form a cyclopropane. Thereafter THP group is removed to obtain the alcohol; subsequently the methyl ester is hydrolyzed to acid and converted into montelukast sodium salt.

Following the same strategy, in WO9518107 an improved process to obtain montelukast is described using the dilithium salt of 1-(mercaptomethyl)cyclopropane acetic acid (Rₐ is H. R_{b} is COOH, R_{c}-R_{d} form a cyclopropane, and L is an aryl- or alkyl-sulfonyl group, e.g. methanesulfonyl (mesyl)). A further improvement of this process is described in WO2004108679. In WO2005105751, the preparation of montelukast is described following this strategy to obtain esters of montelukast (Rₐ is H. R_{b} is COO-alkyl and R_{c}-R_{d} form a cyclopropane). These esters are further hydrolyzed to yield the corresponding carboxylic acid. In US20050234241-A1, R_{b} is CN or CONH₂, an intermediate form (precursor) of the final carboxylic acid present in montelukast. After the coupling, these intermediate forms are then hydrolyzed to yield the carboxylic acid, montelukast, and converted into its sodium salt.

Another route of synthesis was described in EP480717-A1, in Example 15, step 12, for a structurally related compound by using the Wittig reaction (strategy B). In this case n is 0, Rₐ and R_{d} are H. R_{b} is COOMe and R_{c} is a methyl group.

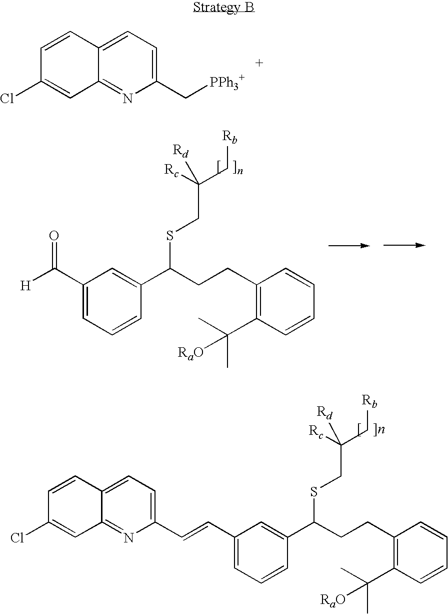

wherein:
Rₐ, R_{b}, R_{c} and R_{d} are as defined in strategy A and n is 0 or 1.

An additional strategy to prepare montelukast comprises the reaction of an ester of a carboxylic acid or a ketone with an organometallic compound, such as MeMgBr or MeLi, to yield the corresponding alcohol (strategy C).

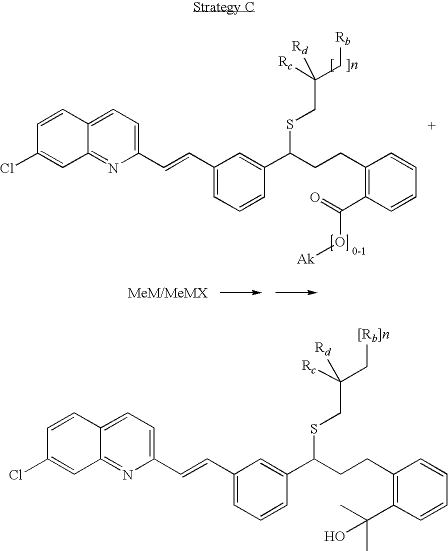

wherein:
R_{b}, R_{c}, R_{d} and n are as described in strategy A, M is a metal, X is a halide and Ak is an alkyl group.

This strategy is followed in EP480717-A1 to prepare certain intermediates (for instance, in Example 16, step 5; Example 15, step 9 and Method C on page

28) and to obtain montelukast and its salts in US20050107612-A and WO2005105750-A1.

Yet another strategy to prepare montelukast comprises the reaction shown below as Strategy D.

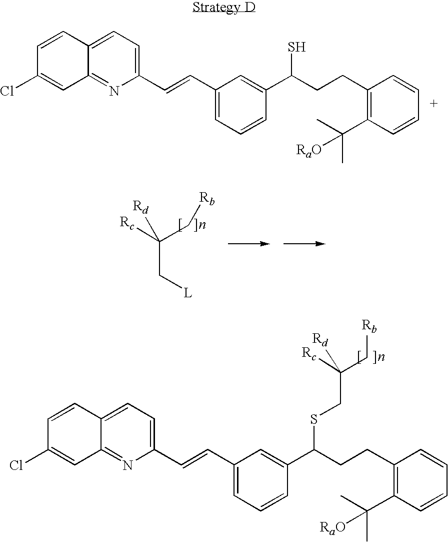

wherein:
Rₐ, R_{b}, R_{c}, R_{d} and n are as defined in strategy A, L is a leaving group.

This strategy is followed in CN1428335-A, CN1420113-A and WO2005105749-A2 for the synthesis of montelukast.

### SUMMARY OF THE INVENTION

The aim of this invention is to provide an efficient alternative process for preparing montelukast, salts thereof, especially its sodium salt, and intermediates for the synthesis of montelukast.

A first aspect of the invention relates to a process for the preparation of a compound of formula (I) or any of its enantiomers or a salt thereof,

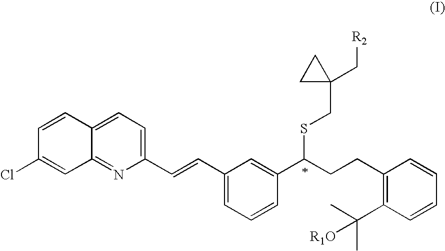

wherein:
R₁ is H or an alcohol protecting group, and
R₂ is COOH or a carboxylic acid intermediate or protected form, that can be transformed into COOH;
comprising the reaction of an intermediate of formula (II),

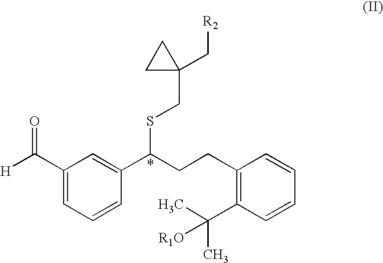

wherein:
R₁ and R₂ have the same meaning as in (I);
with 7-chloro-2-methyl quinoline in an appropriate solvent system and thereafter optionally transforming said R₁ protecting group into H and/or said intermediate or protected forms of R₂ into a carboxylic acid group and, if desired, isolating the R-enantiomer of (I) and, if desired, converting said compound of formula (I) or R-enantiomer thereof to a pharmaceutically acceptable salt thereof.

The inventors have identified a simplified procedure for creating the double bond of the ethenyl moiety without using the Wittig reaction as in Strategy B. The double bond is created through the condensation of an aldehyde and 7-chloro-2-methylquinoline. Novel intermediates are described as well as their preparation.

The main advantage of this procedure, compared with the one described in Strategy B, is its simplicity, by using 7-chloro-2-methylquinoline it is not necessary to functionalize the methyl group. This results in a process with better atomic economy.

Furthermore, the problems of waste, related to the difficulty in removing triphenylphosphine oxide by-product, which occur with Strategy B, are avoided. Reducing the complexity and the cost of preparing active pharmaceutical ingredients is of great interest, especially in the case of montelukast, whose preparation is very complex and involves many steps.

In addition, this process also avoids the low temperatures needed in Strategy B for the formation of the ylide/ylene from the phosphonium salt intermediate.

In a second aspect, the invention relates to compounds of formula (II),

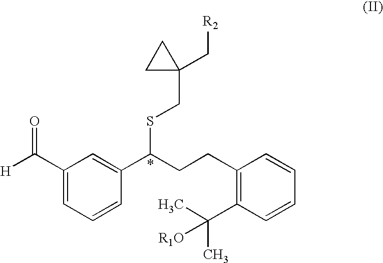

wherein:
R₁ is H or an alcohol protecting group, and
R₂ is COOH or a carboxylic acid intermediate or protected form, that can be transformed into COOH;
provided that R₂ is not COOMe,
which are useful intermediates for the process according to the first aspect of the invention.

A third aspect of the present invention relates to a compound of formula (VI),

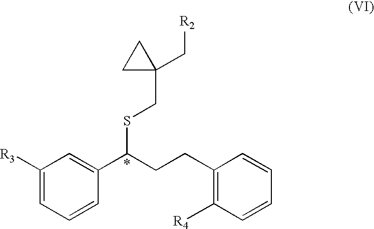

wherein:
R₂ is COOH or a carboxylic acid intermediate or protected form,
R₃ is an aldehyde in a protected form,
R₄ is selected from the group consisting of Br, Cl, 1, —C(CH₃)₂OR₅ and —COOR₆,
R₅ is H or an alcohol protecting group, and
R₆ is a (C₁-C₆)-alkyl group.

These compounds are useful as intermediates for the preparation of compounds of formula (II), which are in turn useful for preparing compounds of formula (I).

A fourth aspect of the present invention relates to compounds of formula (III),

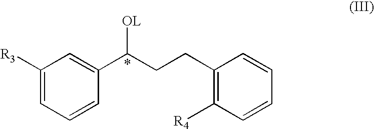

wherein:
R₃ is an aldehyde or an aldehyde in a protected form,
R₄ is selected from the group consisting of Br, Cl, 1, —C(CH₃)₂OR₅ and —COOR₆,
R₅ is H or an alcohol protecting group,
R₆ is a (C₁-C₆)-alkyl group, and
L is an alcohol activating group.

These compounds are useful as intermediates for the preparation of compounds of formula (VI).

A fifth aspect of the present invention relates to compounds of formula (VII),

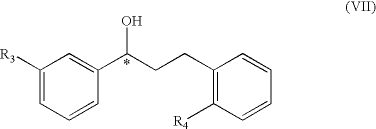

wherein:
R₃ is an aldehyde or an aldehyde in a protected form,
R₄ is selected from the group consisting of Br, Cl, 1, —C(CH₃)₂OR₅ and —COOR₆,
R₅ is H or an alcohol protecting group, and
R₆ is a (C₁-C₆)-alkyl group.

These compounds are useful as intermediates to prepare compounds of formula (III).

A sixth aspect of the present invention relates to compounds of formula (V),

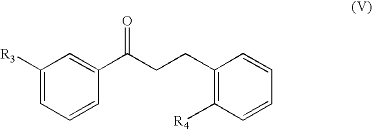

wherein:
R₃ is an aldehyde in a protected form,
R₄ is selected from the group consisting of Br, Cl, I, —C(CH₃)₂OR₅ and —COOR₆,
R₅ is H or an alcohol protecting group, and
R₆ is a (C₁-C₆)-alkyl group.

These compounds are useful as intermediates for the preparation of compounds of formula (VII) and (III).

A further aspect of the invention relates to the use of compounds according to the second to the sixth aspect of the invention for the manufacture of montelukast, salts thereof or montelukast intermediates.

### DEFINITIONS

In the present invention, a carboxylic acid intermediate or protected form is understood as being a group such as a cyano, ester, amide, optionally substituted, or others that can be transformed into a carboxylic acid group by methods well known to a person skilled in the art.

In the present invention an alcohol protecting group is understood as being any protective group of an alcohol of the ether or ester type described, for example, in Greene, T. W. et al., “Protective groups in organic synthesis”, John Wiley and Sons, Third Edition, New York, 1999, hereby incorporated by reference.

In the present invention an alcohol activating group is understood as being a group such as alkyl/aryl sulfonates, e.g. methanesulfonyl (mesyl), toluenesulfonyl (tosyl), etc, that converts the alcohol into a suitable leaving group.

In the present invention an aldehyde in a protected form is understood as being a dialkyl acetal, e.g. dimethyl or diethyl acetal, or cyclic acetals such as 1,3-dioxolanes or 1,3-dioxanes or those described in the literature (e.g. Greene, T. W. et al., “Protective groups in organic synthesis”, John Wiley and Sons, Third Edition, NewYork, 1999).

In the present invention a C₁-C₆alkyl group is understood as being a linear or branched alkyl group which contains up to 6 carbon atoms. Thus it comprises, for instance, methyl, ethyl, propyl, isopropyl, butyl, isobutyl, sec-butyl, tert-butyl, n-pentyl, 1,2-dimethyl propyl, 1,1-dimethyl propyl, 2,2-dimethyl propyl, 2-ethyl propyl, n-hexyl, 1,2-dimethyl butyl, 2,3-dimethyl butyl, 1,3-dimethylbutyl, 1-ethyl-2-methylpropyl, and 1-methyl-2-ethyl propyl groups.

### DETAILED DESCRIPTION OF PARTICULAR EMBODIMENTS

As described above, the invention relates to a process for preparing a compound of formula (I) or any of its enantiomers or a salt thereof, with 7-chloro-2-methylquinoline.

When —OR₁ and/or R₂ in the compounds of formula (I) and (II) are a protected form that can be transformed into a hydroxyl group and/or carboxylic acid group respectively, the process according to the first aspect of the invention further comprises a step in which the protective groups are transformed to obtain the corresponding hydroxyl and/or carboxylic acid moiety. The protective group can be removed by procedures known in the art (e.g. Greene, T. W. et al., “Protective groups in organic synthesis”, John Wiley and Sons, Third Edition, New York, 1999, hereby incorporated by reference).

If R₂ in the compounds of formula (I) and (II) is not a carboxylic acid, the process according to the first aspect of the invention further comprises conversion of said intermediate form to a carboxylic acid. Preferable intermediate forms are an ester, cyano, or an optionally substituted amide. The intermediate form may be converted to the carboxylic acid by methods known by a person skilled in the art. For example, if R₂ is an ester group, it can be hydrolyzed to carboxylic acid under acidic or basic conditions. If, for instance, R₂ is a cyano group it can be converted to the carboxylic acid following the conditions described in ES2114882-T, Example 160, step 4.

The compound of formula (I), obtained by the process according to the first aspect of the invention, may be converted to a pharmaceutically acceptable salt thereof by methods well known by a person skilled in the art.

In another embodiment, the process according to the first aspect of the invention further comprises isolation of the R-enantiomer of the compound of formula (I). The isolation of the R-enantiomer could be carried out by methods known in the art.

The best conditions to carry out the process vary according to the parameters considered by the person skilled in the art, such as solvents, temperature, catalyst and the like. Such reaction conditions may easily be determined by a person skilled in the art using routine tests, and with the teaching of the examples given in this document.

In a more preferred embodiment, the intermediate of formula (II) has R-enantiomeric configuration. Thus, the compound of formula (I) obtained has R-enantiomeric configuration.

Preferably, the process according to the first aspect of the invention is carried out without the use of protecting groups or intermediate forms of the compound of formula (II). Thus, in a preferred embodiment R₁ is hydrogen and R₂ is carboxylic acid.

The reaction between the intermediate of formula (II) and 7-chloro-2-methyl quinoline is preferably carried out in the presence of at least one acid or basic catalyst. In a preferred embodiment, the reaction is carried out in the presence of at least one basic catalyst. Suitable basic catalysts include organic bases such as secondary or tertiary alkyl or cycloalkyl amines.

The reaction may be carried out in different organic solvents. Preferably, the solvent system is an organic solvent such as aromatic apolar solvent or alcohol or mixture thereof. In a preferred embodiment, the reaction is carried out in the presence of toluene or isobutyl alcohol.

The intermediate of formula (II) may be prepared by methods described in the literature (EP0604114-A1, Example 1, step 17). The method described therein comprises an eight step process starting from isophthalaldehyde. The present inventors have also found a new and simplified process for the preparation of an intermediate of formula (II), which may constitute a separate aspect of the invention. Thus, in a preferred embodiment of the invention the intermediate of formula (II) is prepared by reaction between an intermediate of formula (III) in the presence of a base,

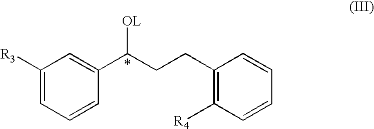

wherein:
R₃ is CHO or an aldehyde in a protected form,
R₄ is selected from the group consisting of Br, Cl, I, —C(CH₃)₂OR₅ and —COOR₆,
R₅ is H or an alcohol protecting group,
R₆ is a (C₁-C₆)-alkyl group, and
L is an alcohol activating group;
with an intermediate of formula (IV) or a salt thereof,

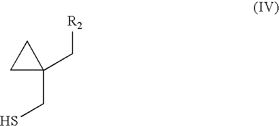

wherein R₂ has the same meaning as in the compound of formula (I);
and if required converting R₄ to —C(CH₃)₂OR₅, and if required converting the aldehyde in a protected form to aldehyde.

When R₃ of the intermediate of formula (III) is a protected form that can be transformed into an aldehyde, the process according to this embodiment further comprises the conversion of said intermediate form to an aldehyde group. If the reactions involved to transform (III) into (II) require R₃ to be protected, the person skilled in the art would understand that R₃ should be restricted to an aldehyde in a protected form. In a preferred embodiment R₃ is protected as a 5,5-dimethyl-1,3-dioxan-2-yl or [1,3]dioxolan-2-yl group. Suitable procedures for the conversion of the protected form to an aldehyde are described, for example, in Greene, T. W. et al., “Protective groups in organic synthesis”, John Wiley and Sons, Third Edition, New York, 1999, hereby incorporated by reference.

In a preferred embodiment, the alcohol activating group L of the intermediate of formula (III) is an alkyl- or aryl-sulfonyl group, preferably methanesulfonyl (mesyl) or para-toluenesulfonyl (tosyl). Moreover, the arylsulfonyl group may be substituted, preferably with a methyl group.

In another embodiment, R₄ of the intermediate of formula (III) is a halogen selected from bromine, chlorine or iodine than can be transformed into 2-hydroxypropan-2-yl or into a protected form of 2-hydroxypropan-2-yl by reaction of the organometallic derivative with acetone as described in Example 7.

In a preferred embodiment, R₄ of the intermediate of formula (III) is an ester that can be transformed into an alcohol as described in the literature (e.g. according to EP480717-A1, Example 16, step 5). For example, if R₄ is COOR₆, being R₆ a (C₁-C₆)-alkyl group, it can be transformed into an alcohol by reaction with CH₃M or CH₃MX, where M is a metal and X is a halogen. More preferably R₄ is —COOMe.

In a more preferred embodiment, the intermediate of formula (III) has S-enantiomeric configuration. Thus, the compound of formula (II) and (I) obtained have R-enantiomeric configuration.

An additional embodiment of the invention relates to a process for preparing an intermediate of a compound of formula (III) wherein it is prepared by reduction of an intermediate of formula (V),

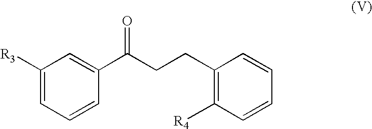

wherein:
R₃ is an aldehyde in a protected form and R₄ has the same meaning as in the compound of formula (III); to give the corresponding alcohol which is then converted into intermediate (III) by introduction of an alcohol activating group and optionally, R₃ is converted into an aldehyde group if desired.

In a preferred embodiment R₃ is protected as a 5,5-dimethyl-1,3-dioxan-2-yl or [1,3]dioxolan-2-yl group and in a more preferred embodiment R₄ is —COOR₆ wherein R₆ is a (C₁-C₆)-alkyl group.

Different reducing agents may be appropriate for the reaction. Preferably, the reducing agent is stereoselective. Even more preferably, the stereoselective reducing agent affords the alcohol in (S)-configuration.

A variety of alcohol activating groups may be used in the process. Preferably, the activation takes place with an alkyl- or aryl-sulfonyl halide, such as mesyl halide or tosyl halide. Even more preferably, it takes place with mesyl chloride.

The intermediate of formula (V) may be obtained by reacting 3-(2-bromophenyl)-propionaldehyde with 2-(3-bromophenyl)-[1,3]dioxolane by a Grignard reaction, followed by an oxidation of the alcohol thus obtained to form a ketone of formula (III). The intermediate 3-(2-bromophenyl) propionaldehyde may be prepared by methods described in the literature (e.g. Cooke, M. P. et al., J. Org. Chem (1987), 52 (8), 1381-1396).

The second aspect of the present invention relates to compounds of formula (II) which are useful as intermediates in the synthesis of montelukast and related compounds. R₂ is preferably COOH, an ester, cyano or amide group, optionally substituted. More preferably R₂ is COOH. In a preferred embodiment R₁ is H and R₂ is COOH. In a more preferred embodiment, compound of formula (II) has R-enantiomeric configuration.

The third aspect of the invention relates to compounds of formula (VI) which are useful as intermediates in the synthesis of montelukast and related compounds. In one embodiment, R₂ is COOH, R₃ is an aldehyde protected as 5,5-dimethyl-1,3-dioxan-2-yl and R₄ is —C(CH₃)₂OH. In another embodiment, R₂ is COOH, R₃ is an aldehyde protected as 5,5-dimethyl-1,3-dioxan-2-yl and R₄ is —COOR₆, wherein R₆ is a (C₁-C₆)-alkyl group, preferably methyl. In a preferred embodiment, the compound of formula (VI) has R-enantiomeric configuration.

The fourth aspect of the invention relates to compounds of formula (III) which are useful as intermediates in the synthesis of montelukast and related compounds. In one embodiment, R₃ is an aldehyde protected as 5,5-dimethyl-1,3-dioxan-2-yl or [1,3]dioxolan-2-yl, R₄ is —COOR₆, wherein R₆ is a (C₁-C₆)-alkyl group, and L is an alcohol activating group, preferably an alkyl- or aryl-sulfonyl group, optionally substituted. Preferably, the alcohol activating group is an alkylsulfonyl group. Even more preferably, R₃ is 5,5-dimethyl-1,3-dioxan-2-yl, R₄ is —COOMe and L is methanesulfonyl. In a further preferred embodiment, the compound of formula (III) has S-enantiomeric configuration.

The fifth aspect of the invention relates to compounds of formula (VII) which are useful as intermediates for the preparation of compounds of formula (III). In one embodiment, R₃ is an aldehyde protected as 5,5-dimethyl-1,3-dioxan-2-yl or [1,3]dioxolan-2-yl group and R₄ is —COOR₆, wherein R₆ is a (C₁-C₆)-alkyl group. In a preferred embodiment, R₃ is 5,5-dimethyl-1,3-dioxan-2-yl and R₄ is —COOMe. In a more preferred embodiment, the compound of formula (VII) has S-enantiomeric configuration.

The sixth aspect of the invention provides compounds of formula (V) which are useful as intermediates in the synthesis of montelukast and related compounds. In a preferred embodiment, R₃ is an aldehyde protected as 5,5-dimethyl-1,3-dioxan-2-yl or [1,3]dioxolan-2-yl and R₄—COOR₆, wherein R₆ is a (C₁-C₆)-alkyl group. In a preferred embodiment, R₃ is 5,5-dimethyl-1,3-dioxan-2-yl and R₄ is —COOMe.

The purification of all intermediates and final products by methods known in the art should be considered as included in the scope of the invention. One of the standard purification methods is the preparation of intermediates in its solid state, preferably in crystalline form by conventional crystallisation and recrystallisation techniques using solvents that a person skilled in the art considers to be the most suitable.

Throughout the description and claims the word “comprise” and variations of the word, such as “comprising”, are not intended to exclude other technical features, additives, components, or steps. Additional objects, advantages and features of the invention will become apparent to those skilled in the art upon examination of the description or may be learned by practice of the invention. The following examples are provided by way of illustration, and are not intended to be limiting of the present invention.

### EXAMPLES

### Example 1

### 3-(2-bromophenyl)propionaldehyde

Following the procedure described by Stambuli, J. P. in J. Am. Chem. Soc. (2001), 123 (11), 2677-2678, for the reduction of 3-(4-bromophenyl)propionic acid, 3-(2-bromophenyl)propionic acid was reduced to 3-(2-bromophenyl)propan-1-ol using BH₃.SMe₂ in tetrahydrofurane. Then the alcohol was converted to the corresponding aldehyde following the procedure described by Cooke, M. P. in J. Org. Chem. (1987), 52 (8), 1381-1396.

### Example 2

### 3-(2-bromophenyl)-1-(3-[1,3]dioxolan-2-yl-phenyl)propan-1-ol

A mixture of magnesium turnings (1.17 g, 48.34 mmol) in tetrahydrofurane (5 mL) was charged in a 100 mL three-necked flask equipped with a condenser and a dropping funnel under argon. A crystal of iodine was added and the mixture was then treated with a solution of 2-(3-bromophenyl)-[1,3]dioxolane (6.9 mL, 45.60 mmol) in tetrahydrofurane (15 mL) via the dropping funnel. An exothermic reaction was initiated and the reaction mixture refluxed moderately. The resultant grey mixture was agitated for 0.5 h. A solution of 3-(2-bromophenyl) propionaldehyde (9.53 g, 44.76 mmol) in tetrahydrofurane (28 mL) was placed in the dropping funnel and was slowly added to the reaction mixture. A refluxing brown solution was obtained. The reaction mixture was stirred for 2 hours and then quenched with a saturated aqueous solution of ammonium chloride (80 mL). Then 15 mL of water were added. The organic layer was separated and the aqueous phase extracted with tetrahydrofurane (45 mL). The combined organic phases were dried over sodium sulfate and concentrated. Flash chromatography using cyclohexane:ethyl acetate mixtures afforded the title compound as a yellow oil (11.040 g, 68%).

¹H-NMR (400 MHz, CDCl₃) 6 (ppm): 7.50 (m, 2H), 7.38 (m, 3H), 7.22 (m, 2H), 7.05 (m, 1H), 5.80 (s, 1H), 4.75 (t, 1H), 4.13 and 4.03 (2 m, 4H), 2.85 (m, 2H), 2.06 (m, 2H).

¹³C-NMR (100 MHz, CDCl₃) 6 (ppm): 144.68, 141.08, 138.10, 132.80, 130.40, 128.58, 127.60, 127.42, 126.79, 125.83, 124.42, 123.93, 103.65, 73.73, 65.30, 38.79, 32.54.

### Example 3

### 3-(2-Bromophenyl)-1-(3-[1,3]dioxolan-2-yl-phenyl)propan-1-one

A solution of 3-(2-bromophenyl)-1-(3-[1,3]dioxolan-2-yl-phenyl)propan-1-ol (5.37 g, 14.8 mmol) in 27 mL of dichloromethane was added to a stirred mixture of pyridinium chlorochromate (3.82 g, 17.8 mmol) in 27 mL of dichloromethane under argon. After 3 hours 125 mL of diethyl ether were added and the solution was decanted from the chromium salts. The residue was extracted twice with 54 mL of diethyl ether and the combined organic phases were filtered through a 1-cm plug of silica gel on a glass microfibre filter. The dark solution was concentrated and the residue was purified by flash chromatography using 9:1 cyclohexane-ethyl acetate. The title compound was obtained as yellow oil (4.176 g, 78%).

¹H-NMR (400 MHz, CDCl₃) δ (ppm): 8.08 (s, 1H), 7.98 (d, 1H), 7.67 (d, 1H), 7.54 (d, 1H), 7.46 (t, 1H), 7.30 (d, 1H), 7.23 (t, 1H), 7.07 (t, 1H), 5.83 (s, 1H), 4.12 and 4.03 (2 m, 4H), 3.32 (t, 2H, 3.17 (t, 2H).

¹³C-NMR (100 MHz, CDCl₃) δ (ppm): 198.43, 140.43, 138.58, 136.80, 132.80, 131.14, 130.72, 128.71, 128.66, 127.91, 127.56, 126.15, 124.27, 103.05, 65.30, 38.63, 30.68.

### Example 4

### (S)-3-(2-bromophenyl)-1-(3-[1,3]dioxolan-2-yl-phenyl)propan-1-ol

To a solution of 0.300 g (0.83 mmol) of 3-(2-bromophenyl)-1-(3-[1,3]dioxolan-2-yl-phenyl)propan-1-one in 3 mL of dry tetrahydrofurane, 165 μL of 1.0 M (R)-tetrahydro-1-methyl-3,3-diphenyl-1H,3H-pyrrolo[1,2-c][1,3,2]oxazaborole (0.17 mmol) were added dropwise. A solution of BH₃.SMe₂ (1.24 mmol) in 1 mL of dry tetrahydrofurane was then slowly added. The mixture was stirred for 30 minutes and then carefully quenched by addition of 10% aqueous diethanolamine. Then 25% aqueous NH₄OAc was added, the layers were separated and the aqueous phase was extracted with ethyl acetate. The combined organic layers were dried over sodium sulfate and the solvent evaporated in vacuo. Flash chromatography using cyclohexane:ethyl acetate mixtures afforded the title compound as a light yellow oil (0.271 g, 90%). Chiral purity: 92.4% enantiomeric excess.

### Example 5

### {1-[3-(2-bromophenyl)-1-(3-[1,3]dioxolan-2-yl-phenyl)propylsulfanyl-methyl]cyclopropyl}acetic acid

In a 100 mL two-necked flask 5.59 g (15.4 mmol) of 3-(2-bromophenyl)-1-(3-[1,3]-dioxolan-2-yl-phenyl)propan-1-ol were dissolved in 56 mL of dichloromethane under argon. Then 3.8 mL (27.04 mmol) of triethylamine were added and the mixture was cooled to −20° C. Then 1.55 mL of methanesulfonyl chloride were added dropwise. The mixture was stirred for 20 minutes and then treated with a saturated aqueous solution of sodium hydrogen carbonate (65 mL). The organic layer was separated and the aqueous phase extracted with dichloromethane (50 mL). The combined organic layers were dried with sodium sulfate and concentrated. The corresponding mesylate was obtained as a pale yellow oil.

In a 250 mL three-necked flask equipped with a dropping funnel, a solution was prepared by dissolving 2.36 g (16.17 mmol) of 2-[1-(mercaptomethyl)cyclopropyl]acetic acid in 65 mL of anhydrous tetrahydrofurane. The solution was cooled to −15° C. and 17 mL of BuLi 1.92 M (32.64 mmol) were added dropwise via the dropping funnel. After 45 minutes, a solution of the mesylate in 39 mL of dry tetrahydrofurane was placed in the dropping funnel and added slowly. The resultant mixture was stirred at −5° C. for 4 hours and then quenched with 10 mL of water. The solvent was evaporated and the residue partitioned between 100 mL of toluene and 100 mL of aqueous 10% sodium carbonate. The organic layer was separated and the aqueous phase extracted with 50 mL of toluene. The combined organic layers were discarded. The aqueous phase was then acidified with 250 mL of a 0.5 M aqueous solution of tartaric acid and extracted with 100 mL of toluene. The aqueous phase was extracted again with toluene (50 mL) and the combined organic layers were dried over sodium sulfate and concentrated. The title compound was obtained as a yellow oil (4.351 g, 58%).

¹H-NMR (400 MHz, CDCl₃) δ (ppm): 7.49 (d, 1H), 7.45 (s, 1H), 7.35 (m, 3H), 7.16 (m, 2H), 7.03 (t, 1H), 5.80 (s, 1H), 4.15 and 4.05 (2 m, 4H), 3.83 (t, 1H), 2.79 and 2.67 (2 m, 2H), 2.46 (s, 2H), 2.45 and 2.29 (2d, 2H), 2.14 (m, 2H), 0.47 (m, 4H).

¹³C-NMR (100 MHz, CDCl₃) δ (ppm): 176.60, 142.71, 140.76, 137.72, 132.82, 130.43, 128.93, 128.62, 127.69, 127.39, 126.24, 125.49), 124.34, 103.77, 65.31, 65.27, 49.67, 39.82, 38.53, 36.56, 34.41, 16.54, 12.46, 12.27.

### Example 6

### R-{1-[3-(2-bromophenyl)-1-(3-[1,3]dioxolan-2-yl-phenyl)propylsulfanyl-methyl]cyclopropyl}acetic acid

Following the procedure of Example 5 with (S)-3-(2-bromophenyl)-1-(3-[1,3]-dioxolan-2-yl-phenyl)propan-1-ol affords the title compound.

### Example 7

### (1-{1-(3-[1,3]dioxolan-2-yl-phenyl)-3-[2-(1-hydroxy-1-methylethyl)phenyl]-propylsulfanylmethyl}cyclopropyl)acetic acid

A solution of 2.00 g (4.07 mmol) of {1-[3-(2-bromophenyl)-1-(3-[1,3]-dioxolan-2-yl-phenyl)propylsulfanylmethyl]cyclopropyl}acetic acid in 20 mL of dry tetrahydrofurane was cooled to −94° C. and then 4.7 mL of BuLi 1.83 M (8.60 mmol) were added slowly. The resultant red solution was agitated for ten minutes and 0.49 mL of freshly distilled dry acetone were added dropwise. The mixture was stirred at −90° C. for 30 minutes and then allowed to warm slowly to room temperature. After 1.5 hours the reaction mixture was treated with 20 mL of 0.5 M aqueous solution of tartaric acid. The layers were separated and the aqueous phase was extracted with 20 mL of dichloromethane. The combined organic layers were dried with sodium sulfate and the solvent evaporated in vacuo. The residue obtained was purified by flash chromatography using cyclohexane:ethyl acetate mixtures. The title compound was obtained as yellow oil (0.893 g, 47%).

¹H-NMR (400 MHz, CDCl₃) δ (ppm): 7.47 (s, 1H), 7.34 (m, 4H), 7.14 (m, 3H), 5.79 (s, 1H), 4.15 and 4.04 (2 m, 4H), 3.94 (t, 1H), 3.10, 2.85 (2 m, 2H), 2.52-2.28 (m, 4H), 2.17 (m, 2H), 1.58 and 1.57 (2 s, 6H), 0.46 (m, 4H).

¹³C-NMR (100 MHz, CDCl₃) δ (ppm): 176.84, 145.08, 143.11, 140.15, 137.64, 131.43, 128.97, 128.44, 127.02, 126.21, 125.52, 125.34, 125.29, 103.75, 73.65, 65.22, 50.18, 40.01, 39.59, 38.77, 32.19, 31.73, 16.64, 12.61, 12.16.

### Example 8

### R-(1-{1-(3-[1,3]dioxolan-2-yl-phenyl)-3-[2-(1-hydroxy-1-methylethyl)phenyl]-propylsulfanylmethyl}cyclopropyl)acetic acid

Following the procedure of Example 7 with R-{1-[3-(2-bromophenyl)-1-(3-[1,3]-dioxolan-2-yl-phenyl)propylsulfanylmethyl]cyclopropyl}acetic acid affords the title compound.

### Example 9

### (1-{1-(3-formyl-phenyl)-3-[2-(1-hydroxy-1-methyl-ethyl)-phenyl]-propylsulfanylmethyl}-cyclopropyl)-acetic acid

A solution of (1-{1-(3-[1,3]dioxolan-2-yl-phenyl)-3-[2-(1-hydroxy-1-methyl-ethyl)-phenyl]-propylsulfanylmethyl}-cyclopropyl)-acetic acid (0.996 g, 2.12 mmol) and p-toluenesulfonic acid (28.2 mg, 0.15 mmol) in 10 mL of tetrahydrofurane:water 1:1 was heated to 50° C. under argon for 3 hours. Tetrahydrofurane was evaporated and the aqueous phase extracted twice with 10 mL of ethyl acetate. The combined organic layers were dried over sodium sulfate and concentrated. The corresponding aldehyde was obtained as a yellow oil (0.893 g, 99%).

¹H-NMR (400 MHz, CDCl₃) δ (ppm): 10.00 (S, 1H), 7.88 (s, 1H), 7.75 (d, 1H), 7.68 (d, 1H), 7.49 (t, 1H), 7.33 (d, 1H), 7.13 (m, 3H), 4.02 (t, 1H), 3.13 and 2.86 (2 m, 2H), 2.43 (m, 4H), 2.17 (m, 2H), 1.60 and 1.59 (2 s, 6H), 0.46 (m, 4H).

¹³C-NMR (100 MHz, CDCl₃) δ (ppm): 192.70, 177.53, 145.35, 144.74, 140.20, 136.79, 134.33, 131.67, 129.49, 129.29, 128.89, 127.40, 125.95, 125.71, 73.84, 49.87, 39.91, 39.67, 39.03, 32.23, 31.73, 16.64, 12.77, 12.27.

### Example 10

### R-(1-{1-(3-formyl-phenyl)-3-[2-(1-hydroxy-1-methyl-ethyl)-phenyl]-propylsulfanylmethyl}-cyclopropyl)-acetic acid

Following the procedure of Example 9 with R-(1-{1-(3-[1,3]dioxolan-2-yl-phenyl)-3-[2-(1-hydroxy-1-methyl-ethyl)-phenyl]-propylsulfanylmethyl}-cyclopropyl)-acetic acid affords the title compound.

### Example 11

### (E)-1-[[[1-[3-[2-[7-chloro-2-quinolinyl]ethenyl]phenyl]-3-[2-(1-hydroxy-1-methylethyl)phenyl]propyl]thio]methyl]cyclopropaneacetic acid

A solution of (1-{1-(3-formylphenyl)-3-[2-(1-hydroxy-1-methylethyl)phenyl]propylsulfanylmethyl}cyclopropyl)acetic acid (0.329 g, 0.77 mmol), 7-chloro2-methylquinoline (0.137 g, 0.77 mmol) and piperidine (38 μL, 0.38 mmol) in 3 mL of toluene was refluxed under argon for 25 hours. The solvent was evaporated and the residue partitioned between 5 mL of ethyl acetate and 5 mL of 0.5 M aqueous solution of tartaric acid. The organic layer was separated and the aqueous phase extracted twice with 3 mL of ethyl acetate. The combined organic extracts were dried over sodium sulfate and concentrated. The residue was purified by flash chromatography using cyclohexane:ethyl acetate mixtures and the title compound was obtained as a yellow solid (0.130 g, 29%).

¹H-NMR (400 MHz, CDCl₃) δ (ppm): 8.06 (m, 2H), 7.67 (m, 4H), 7.44 (m, 3H), 7.33 (m, 3H), 7.15 (m, 3H), 4.00 (t, 1H), 3.16 and 2.90 (2 m, 2H), 2.64-2.35 (m, 4H), 2.20 (m, 2H), 1.60 and 1.59 (2 s, 6H), 0.49 (m, 4H).

¹³C-NMR (100 MHz, CDCl₃) δ (ppm): 176.14, 156.91, 148.00, 145.18, 143.54, 140.13, 136.44, 136.39, 135.76, 135.54, 131.46, 128.97, 128.65, 128.59, 128.39, 128.38, 127.48, 127.22, 127.10, 126.56, 126.43, 125.59, 125.36, 119.10, 73.79, 50.28, 40.18, 39.88, 38.84, 32.22, 31.74, 31.73, 16.72, 12.59, 12.32.

### Example 12

### R-(E)-1-[[[1-[3-[2-[7-chloro-2-quinolinyl]ethenyl]phenyl]-3-[2-(1-hydroxy-1-methylethyl)phenyl]propyl]thio]methyl]cyclopropaneacetic acid

Following the procedure of Example 11 with R-(1-{1-(3-formylphenyl)-3-[2-(1-hydroxy-1-methylethyl)phenyl]propylsulfanylmethyl}cyclopropyl)acetic acid affords the title compound.

### Example 13

### 1-(3-(1,3-dioxolan-2-yl)phenyl)ethanone

An 8 mL solution of 100 g (0.44 mol) of 2-(3-bromophenyl)-[1,3]dioxolane in 160 mL of dry tetrahydrofurane was added to a mixture of 11 g (0.46 mol) of magnesium turnings in 30 mL of dry tetrahydrofurane. After adding 0.05 g of iodide, the reaction was left stirring at room temperature until an exothermic reaction was initiated. At this point, the rest of 2-(3-bromophenyl)-[1,3]dioxolane solution was added dropwise maintaining the reaction temperature at 35-40° C. The mixture was left for 2 hours at room temperature and then added dropwise to a cooled solution of 83 mL (0.88 mols) of acetic anhydride in 85 mL of dry tetrahydrofurane at −10° C. After 1 hour at −5° C. the reaction mixture was left to warm to room temperature and then poured into cold saturated aqueous NaHCO₃ solution. The resultant mixture was extracted twice with 300 mL of ethyl acetate and the residue obtained after removing the ethyl acetate was distilled under vacuum (0.9 mbar) to obtain a pure fraction (110-116° C.) of 72 g (85.7%) of the title compound.

¹H-NMR (400 MHz, CDCl₃) δ (ppm): 8.05 (s, 1H), 7.94 (d, 1H), 7.66 (d, 1H), 7.47 (m, 1H), 5.84 (s, 1H), 4.0-4.2 (m, 4H), 2.59 (s, 3H).

### Example 14

### Methyl 3-(3-(1,3-dioxolan-2-yl)phenyl)-3-oxopropanoate

A solution of 100 g (0.52 mol) of 1-(3-(1,3-dioxolan-2-yl)phenyl)ethanone in 500 mL of dry dimethylformamide was added dropwise to a cooled mixture of 26 g (0.65 mol) of 60% NaH dispersed in mineral oil in 150 mL of dry dimethylformamide. It was then stirred for 1 h at 0° C. and then 1 h at room temperature. The mixture was cooled to −10° C. and a solution of 48 mL (0.57 mol) of dimethyl carbonate in 80 mL of dry dimethylformamide was added dropwise maintaining the reaction temperature at 0 to −10° C. After 1 h at 0° C., the reaction mixture was warmed to room temperature and left stirring for a further 3 hours. The mixture was treated with NH₄Cl aqueous solution and extracted three times with 500 mL of ethyl acetate. The combined organic phases were washed with water and after drying with anhydrous sodium sulfate, the solvent was removed by vacuum distillation. The residue was treated with mixture of methanol and n-heptane, the methanol phase was separated and distilled under vacuum to obtain 120.8 g (92%) of title compound as red oil.

¹H-NMR (400 MHz, CDCl₃) δ (ppm): 8.04 (s, 1H), 7.93 (d, 1H), 7.70 (m, 1H), 7.50 (m, 1H), 5.83 (s, 1H), 4.01-4.16 (m, 4H), 4.01 (s, 2H), 3.74 (s, 3H).

### Example 15

### Methyl 2-(2-(3-(1,3-dioxolan-2-yl)benzoyl)-3-methoxy-3-oxopropyl)benzoate

A solution of 37 g (0.148 mol) of methyl 3-(3-(1,3-dioxolan-2-yl)phenyl)-3-oxo-propanoate in 60 mL of dry dimethylformamide was added dropwise to a mixture of 6.21 g (0.155 mol) of 60% NaH dispersed in mineral oil in 250 mL of dimethylformamide at −10° C. The reaction mixture was stirred at 0° C. for 1 hour and then at room temperature for 1 hour. A solution of 33.8 g (0.148 mol) of methyl 2-(bromomethyl)benzoate in 60 mL of dry dimethylformamide was added dropwise maintaining the reaction temperature at between 25 and 40° C. and then the reaction mixture was left stirring at room temperature for 1 hour and then poured into 500 mL of cold saturated NH₄Cl. The reaction mixture was extracted twice with 300 mL of ethyl acetate and the combined organic phases were washed several times with water. After drying with anhydrous sodium sulfate the solvent was distilled in vacuo to obtain a residue that was treated with 40 mL of methanol and 15 mL of n-heptane. After removing the upper layer, the methanol was distilled at in vacuo pressure to obtain crude methyl 2-(2-(3-(1,3-dioxolan-2-yl)benzoyl)-3-methoxy-3-oxopropyl)benzoate.

¹H-NMR (400 MHz, CDCl₃) 6 (ppm): 8.05 (s, 1H), 7.89 (m, 2H), 7.62 (d, 1H), 7.40 (t, 1H), 7.25 (m, 1H), 7.21 (m, 2H), 5.78 (s, 1H), 4.97 (t, 1H), 4.06 (m, 2H), 4.00 (m, 2H), 3.85 (s, 3H), 3.65 (m, 2H), 3.60 (s, 3H).

### Example 16

### Methyl 2-(3-(3-formylphenyl)-3-oxopropyl)benzoate

A 212 mL (2.5 mol) solution of HCl 37% was added to a mixture of 100 g (0.251 mol) of methyl 2-(2-(3-(1,3-dioxolan-2-yl)benzoyl)-3-methoxy-3-oxopropyl)benzoate in 200 mL of water and 500 mL of 1,4-dioxane. The reaction was heated to reflux and controlled by TLC (ethyl acetate/petroleum ether 1:5) until decarboxilation was complete. The reaction mixture was cooled to room temperature and extracted with a mixture of ethyl acetate/petroleum ether 1:2. The combined organic phases where washed with water and saturated NaHCO₃ aqueous solution. The organic phase was dried with anhydrous sodium sulfate and the solvents where removed by vacuum distillation to obtain 60 g (80%) of title compound as yellow oil.

¹H-NMR (400 MHz, CDCl₃) δ (ppm): 10.04 (s, 1H), 8.44 (s, 1H), 8.22 (d, 1H), 8.03 (d, 1H), 7.91 (d, 1H), 7.60 (t, 1H), 7.43 (m, 1H), 7.33 (m, 1H), 7.26 (m, 1H), 3.87 (s, 3H), 3.37 (m, 4H).

### Example 17

### Methyl 2-(3-(3-(5,5-dimethyl-1,3-dioxan-2-yl)phenyl)-3-oxopropyl)benzoate

1.78 g (9.36 mmol) of p-toluenesulfonic acid and 10.77 g (0.103 mol) of neopentylglycol were added successively to a mixture of 30.6 g (0.103 mol) of methyl 2-(3-(3-formylphenyl)-3-oxopropyl)benzoate in 350 mL of toluene. The mixture was heated at reflux for 2 hours and after cooling to room temperature, 80 mL of saturated aqueous NaHCO₃ were added and the mixture was left stirring for 10 min. The organic phase was separated, diluted with 100 mL of ethyl acetate and washed with saturated aqueous NaHCO₃ and water. The organic phase was dried with magnesium sulfate and the solvent was removed by vacuum distillation to obtain a residue that was purified by column chromatography to obtain 21 g (54%) of the title compound.

¹H-NMR (400 MHz, CDCl₃) δ (ppm): 8.10 (s, 1H), 7.99 (d, 1H), 7.94 (d, 1H), 7.73 (d, 1H), 7.44-7.50 (m, 2H), 7.37 (d, 1H), 7.30 (m, 1H), 5.44 (s, 1H), 3.90 (s, 3H), 3.79 (d, 2H), 3.67 (d, 2H), 3.38 (s, 4H), 1.30 (s, 3H), 0.82 (s, 3H).

### Example 18

### 2-((S)-3-hydroxy-3-(3-(5,5-dimethyl-1,3-dioxan-2-yl)phenyl)propyl)benzoate

A solution of 3.5 mL of BH₃.SMe₂ (90% in SMe₂, 33.21 mmol) in 12 mL of toluene was added dropwise to a solution of 1.5 mL of (R)-tetrahydro-1-methyl-3,3-diphenyl-1H,3H-pyrrolo[1,2-c][1,3,2]oxazaborole (1.0 M in toluene, 1.50 mmol) in 26 mL of toluene. Then a solution of methyl 2-(3-(3-(5,5-dimethyl-1,3-dioxan-2-yl)phenyl)-3-oxopropyl)benzoate (8.00 g, 20.92 mmol) in 21 mL of toluene was added dropwise over 1 hour. The mixture was stirred for 30 minutes at room temperature and then carefully quenched at 5° C. by addition of 55 mL of methanol. Then 125 mL of water were added, the layers were separated and the aqueous phase was extracted twice with 100 mL of toluene. The combined organic layers were washed with 100 mL of brine, dried over magnesium sulfate and the solvent evaporated in vacuo. The title compound was obtained as colourless oil (8.08 g, quantitative yield). Chiral purity: 93.7% enantiomeric excess.

¹H-NMR (400 MHz, CDCl₃) δ (ppm): 7.84 (dd, 1H), 7.49 (s, 1H), 7.42 (m, 2H), 7.35 (m, 2H), 7.25 (m, 2H), 5.38 (s, 1H), 4.71 (dd, 1H), 3.87 (s, 3H), 3.76 (d, 2H), 3.64 (d, 2H), 3.09 (t, 2H), 2.06 (m, 2H), 1.29 (s, 3H) and 0.80 (s, 3H).

### Example 19

### (S)-3-(2-(methoxycarbonyl)phenyl)-1-(3-(5,5-dimethyl-1,3-dioxan-2-yl)phenyl)propyl methanesulfonate

In a 100 mL three-necked flask equipped with a magnetic stirrer, a thermometer and a dropping funnel 8.816 g (22.93 mmol) of methyl 2-((S)-3-hydroxy-3-(3-(5,5-dimethyl-1,3-dioxan-2-yl)phenyl)propyl)benzoate were dissolved in 75 mL of dichloromethane under argon. Then 5.0 mL (35.58 mmol) of triethylamine were added and the mixture was cooled to 0-5° C. Then 2.1 mL (27.13 mmol) of methanesulfonyl chloride were added dropwise while keeping the internal temperature below 10° C. The mixture was stirred for 30 minutes at room temperature and then treated with 80 mL of water. The organic layer was separated and washed with a saturated aqueous solution of sodium hydrogen carbonate (50 mL). The organic layer was dried with sodium sulfate and concentrated. The corresponding mesylate was obtained as yellow oil (10.852 g, quantitative yield).

¹H-NMR (400 MHz, CDCl₃) δ (ppm): 7.90 (dd, 1H), 7.54 (m, 2H), 7.41 (m, 3H), 7.26 (m, 2H), 5.60 (dd, 1H), 5.39 (s, 1H), 3.85 (s, 3H), 3.77 (d, 2H), 3.64 (d, 2H), 3.15 and 3.00 (2 m, 2H), 2.64 (s, 3H), 2.38 and 2.20 (2 m, 2H), 1.28 (s, 3H) and 0.80 (s, 3H).

### Example 20

### 2-(1-((((R)-3-(2-(methoxycarbonyl)phenyl)-1-(3-(5,5-dimethyl-1,3-dioxan-2-yl)phenyl)propyl)sulfanyl)methyl)cyclopropyl)acetic acid

In a 250 mL three-necked flask equipped with a magnetic stirrer, a thermometer and a dropping funnel, a solution was prepared by dissolving 3.420 g (23.39 mmol) of 2-(1-(mercaptomethyl)cyclopropyl)acetic acid in 37 mL of dry dimethylformamide. The solution was cooled to −10° C. and 47 mL of lithium bis(trimethylsilyl)amide 1.0 M in tetrahydrofurane (47.00 mmol) were added dropwise via the dropping funnel while keeping the internal temperature below 5° C. The brown solution was stirred at 5° C. for 30 minutes. Then a solution of (S)-3-(2-(methoxycarbonyl)phenyl)-1-(3-(5,5-dimethyl-1,3-dioxan-2-yl)phenyl)propyl methanesulfonate (10.60 g, 22.93 mmol) in 16 mL of dry dimethylformamide was placed in the dropping funnel and added slowly while maintaining the temperature below 5° C. The resultant mixture was stirred at 5° C. for 15 hours and then treated with 120 mL of a 0.5 M aqueous solution of tartaric acid and 120 mL of toluene. Due to the presence of salts, 60 mL of water were added and the mixture was slightly heated. The organic layer was washed four times with 55 mL of water and dried over sodium sulfate. The solvent was evaporated and the title compound was obtained as orange oil (11.76 g, quantitative yield).

¹H-NMR (400 MHz, CDCl₃) δ (ppm): 7.85 (dd, 1H), 7.39 (m, 5H), 7.21 (m, 2H), 5.41 (s, 1H), 3.86 (m, 1H), 3.84 (s, 3H), 3.79 (d, 2H), 3.68 (d, 2H), 3.08 and 2.86 (2 m, 2H), 2.43 (m, 3H), 2.13 (m, 3H), 1.32 (s, 3H), 0.81 (s, 3H), 0.45 (m, 4H).

### Example 21

### 2-(1-((((R)-3-(2-(methoxycarbonyl)phenyl)-1-(3-(5,5-dimethyl-1,3-dioxan-2-yl)phenyl)propyl)sulfanyl)methyl)cyclopropyl)acetic acid

In a 10 mL two-necked flask equipped with a magnetic stirrer, a thermometer and a rubber stopper 0.161 g (1.10 mmol) of 2-(1-(mercaptomethyl)cyclopropyl)acetic acid were dissolved in 1.6 mL of dry dimethylformamide and 220 μL of 15-crown-5 (1.11 mmol) were added. The mixture was cooled to −10° C. and a solution of 0.405 g (2.21 mmol) of sodium bis(trimethylsilyl)amide in 2.2 mL of dry dimethylformamide was added dropwise while keeping the internal temperature below 5° C. The orange solution was stirred at 5° C. for 30 minutes. Then a solution of 0.462 g (1.00 mmol) of (S)-3-(2-(methoxycarbonyl)phenyl)-1-(3-(5,5-dimethyl-1,3-dioxan-2-yl)phenyl)propyl methanesulfonate in 0.9 mL of dry dimethylformamide was added slowly while maintaining temperature below 5° C. The resultant mixture was stirred at 5° C. for 16 hours and then treated with 5 mL of a 0.5 M aqueous solution of tartaric acid, 2.5 mL of water and 5 mL of toluene. The layers were separated and the organic layer was washed with water and dried over sodium sulfate. The solvent was evaporated and the title compound was obtained as orange oil (0.479 g, 94%).

### Example 22

### 2-(1-(((3-(2-(2-hydroxypropan-2-yl)phenyl)-1-(3-(5,5-dimethyl-1,3-dioxan-2-yl)phenyl)propyl)sulfanyl)methyl)cyclopropyl)acetic acid

0.121 g of CeCl₃ (0.49 mmol) and 3.5 mL of dry tetrahydrofurane were transferred to a 10 mL two-necked flask equipped with a magnetic stirrer, a thermometer and a condenser under argon. The suspension was refluxed for 2 hours and then cooled to room temperature. Then 1.65 mL of MeMgCl (3M in tetrahydrofurane, 4.95 mmol) were added dropwise. The mixture was stirred for 45 minutes at room temperature and then cooled to 5-10° C. A solution of 0.503 g (0.98 mmol) of 2-(1-((((R)-3-(2-(methoxycarbonyl)phenyl)-1-(3-(5,5-dimethyl-1,3-dioxan-2-yl)phenyl)propyl)sulfanyl)methyl)cyclopropyl)-acetic acid in 3.2 mL of dry tetrahydrofurane was added dropwise to the reaction mixture while keeping the internal temperature below 10° C. The mixture was stirred at 10° C. for 3 hours and then at room temperature for 1 hour. The reaction was quenched by carefully adding 2M of aqueous AcOH (8 mL). The layers were separated and the aqueous layer extracted twice with 5 mL toluene. The combined organic phases were dried over sodium sulfate and the solvent was removed by vacuum distillation. The title compound was obtained as yellow oil (0.442 g, 88%).

¹H-NMR (400 MHz, CDCl₃) δ (ppm): 7.47 (s, 1H), 7.40-7.07 (m, 7H), 5.40 (s, 1H), 3.93 (t, 1H), 3.78 (d, 2H), 3.67 (d, 2H), 3.05 and 2.87 (2 m, 2H), 2.52-2.28 (m, 4H), 2.17 (m, 2H), 1.58 (s, 3H), 1.56 (s, 3H), 1.31 (s, 3H), 0.81 (s, 3H), 0.47 (m, 4H).

### Example 23

### 2-(1-((((R)-1-(3-formylphenyl)-3-(2-(2-hydroxypropan-2-yl)phenyl) propyl)sulfanyl)methyl)cyclopropyl)acetic acid

A solution of 2.42 g (4.73 mmol) of 2-(1-(((3-(2-(2-hydroxypropan-2-yl)phenyl)-1-(3-(5,5-dimethyl-1,3-dioxan-2-yl)phenyl)propyl)sulfanyl)methyl)-cyclopropyl)acetic acid and 0.549 g (4.73 mmol) of maleic acid in 48 mL of acetone:water (1:1) was heated to 50° C. under argon for 11 hours. Acetone was evaporated and 25 mL of toluene were added. The two phases were separated and the aqueous phase was extracted twice with 5 mL of toluene. The combined organic extracts were dried over sodium sulfate and concentrated. The residue obtained was again dissolved in 48 mL of acetone:water (1:1) and 0.549 g of maleic acid were added. The mixture was stirred at 50° C. under argon for 11 hours and then treated as described above. The obtained residue was purified by flash chromatography using cyclohexane:ethyl acetate: acetic acid mixtures. The title compound was obtained as yellow oil (1.016 g, 50%).

¹H-NMR (400 MHz, CDCl₃) δ (ppm): 10.00 (s, 1H), 7.88 (s, 1H), 7.75 (d, 1H), 7.68 (d, 1H), 7.50 (t, 1H), 7.34 (d, 1H), 7.13 (m, 3H), 4.02 (t, 1H), 3.14 and 2.86 (2 m, 2H), 2.45 (m, 4H), 2.18 (m, 2H), 1.61 (s, 3H), 1.60 (s, 3H), 0.46 (m, 4H).

### Example 24

### R-(E)-1-[[[1-[3-[2-[7-chloro-2-quinolinyl]ethenyl]phenyl]-3-[2-(1-hydroxy-1-methylethyl)phenyl]propyl]thio]methyl]cyclopropaneacetic acid

A solution of 0.985 g (2.31 mmol) of 2-(1-((((R)-1-(3-formylphenyl)-3-(2-(2-hydroxypropan-2-yl)phenyl) propyl)sulfanyl)methyl)cyclopropyl)acetic acid, 0.410 g (2.31 mmol) of 7-chloroquinaldine and 115 μL (1.16 mmol) of piperidine in 10 mL of isobutyl alcohol was refluxed under argon for 13.5 hours and the water-isobutyl alcohol azeotropic mixture was removed by distillation. The loss of solvent was compensated by adding more isobutyl alcohol to the reaction mass. The total volume of distilled isobutyl alcohol-water was 75 mL. Then 10 mL of ethyl acetate and 15 mL of 0.5 M aqueous solution of tartaric acid were added. The organic layer was separated and the aqueous phase extracted twice with 5 mL of ethyl acetate. The combined organic extracts were dried over sodium sulfate and concentrated. The title compound was obtained as orange oil (1.45 g) with a part of unreacted aldehyde and other impurities.

1. A process for the preparation of a compound of formula (I) or any of its enantiomers or a salt thereof, 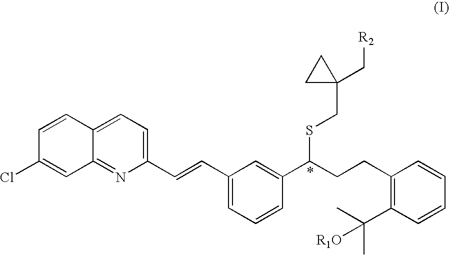
wherein:
R₁ is H or an alcohol protecting group, and
R₂ is COOH or a carboxylic acid intermediate or protected form, that can be transformed into COOH;
comprising the reaction of an intermediate of formula (II), 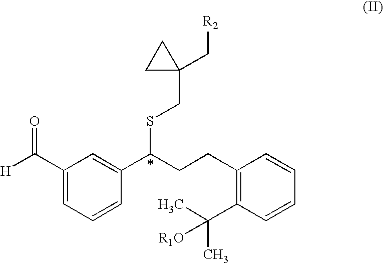
wherein:
R₁ and R₂ have the same meaning as in (1);
with 7-chloro-2-methyl quinoline in an appropriate solvent system and thereafter optionally transforming said R₁ protecting group into H and/or said intermediate or protected forms of R₂ into a carboxylic acid group and, if desired, isolating the R-enantiomer of (I) and, if desired, converting said compound of formula (I) or R-enantiomer thereof to a pharmaceutically acceptable salt thereof.

2. The process according to claim 1, wherein compounds of formula (I) and (II) have R-enantiomeric configuration.

3. The process according to claim 1, wherein R₁, is H and R₂ is COOH.

4. The process according to claim 1, wherein the reaction is carried out in the presence of at least one acid or basic catalyst.

5. The process according to claim 4, wherein the catalyst is selected from the group consisting of secondary or tertiary alkyl or cycloalkyl amines.

6. The process according to claim 1, wherein the reaction takes place in a solvent system comprising an organic solvent selected from an aromatic apolar solvent and an alcohol.

7. The process according to claim 1, wherein intermediate of formula (II) is prepared by the reaction of an intermediate of formula (III) in the presence of a base, 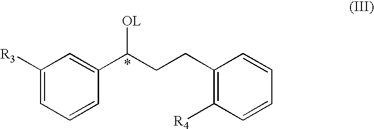
wherein:
R₃ is CHO or an aldehyde in a protected form,
R₄ is selected from the group consisting of Br, Cl, I, —C(CH₃)₂OR₅ and —COOR₆,
R₅ is H or an alcohol protecting group,
R₆ is a (C₁-C₆)-alkyl group, and
L is an alcohol activating group;
with an intermediate of formula (IV) or a salt thereof, 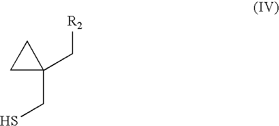
wherein R₂ has the same meaning as in the compound of formula (I);
and if required converting R₄ to —C(CH₃)₂OR₅, and if required converting the aldehyde in a protected form to aldehyde.

8. The process according to claim 7, wherein R₃ is 5,5-dimethyl-1,3-dioxan-2-yl or [1,3]dioxolan-2-yl.

9. The process according to claim 7, wherein L is —SO₂R₇ and R₇ is an alkyl or an aryl group, optionally substituted.

10. The process according to claim 7, wherein L is —SO₂CH₃.

11. The process according to claim 7, wherein R₄ is —COOR₆ and R₆ is a (C₁-C₆)-alkyl group.

12. The process according to claim 7, wherein the compound of formula (III) has S-enantiomeric configuration.

13. The process according to claim 7, wherein intermediate (III) is prepared by reduction of intermediate of formula (V), 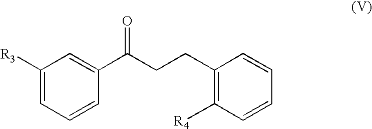
wherein:
R₃ is an aldehyde in a protected form and R₄ has the same meaning as in the compound of formula (III);
to give the corresponding alcohol which is then converted into intermediate (III) by introduction of an alcohol activating group and optionally, R₃ is converted into an aldehyde group if desired.

14. The process according to claim 13, wherein R₃ is 5,5-dimethyl-1,3-dioxan-2-yl or [1,3]dioxolan-2-yl.

15. The process according to claim 13, wherein R₄ is —COOR₆ and R₆ is a (C₁-C₆)-alkyl group.

16. The process according to claim 13, wherein the reducing agent is stereoselective and the alcohol obtained has S-enantiomeric configuration.

17. A compound of formula (II), 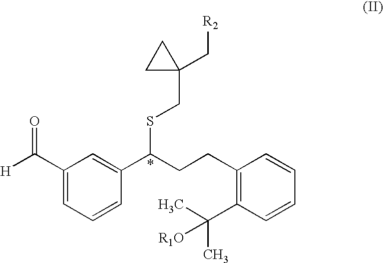
wherein:
R₁ is H or an alcohol protecting group, and R₂ is COOH.

18. The compound of formula (II) according to claim 17, wherein R₁ is H.

19. The compound according to claim 18 having the R-enantiomeric configuration.

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. A method for the manufacture of montelukast, salts thereof or montelukast intermediates, comprising the use of a compound according to claim 17.
